(19) United States
(12) Patent Application Publication
(10) Pub. No.: US 2006/0123800 A1
(43) Pub. Date: Jun. 15, 2006

(54) Desinfection device for a cryostat

(76) Inventor: Rolf Metzner, Dossenheim (DE)

Correspondence Address: HODGSON RUSS LLP, ONE M & T PLAZA, SUITE 2000, BUFFALO, NY, 14203-2391, US

(21) Appl. No.: 11/281,081

(22) Filed: Nov. 17, 2005

(30) Foreign Application Priority Data
Nov. 20, 2004 (DE) 10 2004 056 189.3

(51) Int. Cl. F25B 19/00; F25D 23/00

(52) U.S. Cl. 62/51.1; 62/264

(57) ABSTRACT

A cryostat (1) having a container (2) for receiving a microtome is described, having a cover (3) closing off the container (2) and having a disinfection device associated with which is a control circuit (4). The microtome comprises a knife holder, and the disinfection device is equipped with a UV light source (5).

### CROSS REFERENCE TO RELATED APPLICATIONS

This application claims priority of the German patent application 10 2004 056 189.3 which is incorporated by reference herein.

### FIELD OF THE INVENTION

The present invention relates to a cryostat of a type having a container for receiving a microtome, the microtome comprising a knife holder; the cryostat having a cover for closing off the container, a disinfection device, and a control circuit associated with the disinfection device.

### BACKGROUND OF THE INVENTION

Cryostat microtomes are designed to cool the specimens that are to be cut to a specific predefined temperature. The temperatures are as a rule between −10° C. and −50° C. In order to guarantee temperature consistency, the microtomes are arranged in complex encapsulated housings. A stainless-steel container to receive the microtome is provided in the interior of the housing. The cryostat furthermore comprises a closable viewing window or a cover, through which the user gains access to the container and to the interior space.

Cutting waste also inevitably occurs during microtome operation, and must be removed from the cryostat from time to time. Because the cutting waste that occurs may also be biologically or chemically contaminated, a disinfection of the interior of the housing is also performed upon cleaning of the cryostat.

During this cleaning and disinfection, the cryostat is defrosted and a cleaning and/or disinfection fluid is sprayed with a spray bottle into the interior of the cryostat. This manual method has proven successful, but of course is very time-consuming.

DE 88 14 284 U1 discloses a cryostat that comprises a cutting-waste pan filled with a disinfecting agent. This waste pan extends over only a small region within the cryostat, however, so that complete cleaning and/or disinfection is not possible.

An automatic disinfection system for a cryostat is known from the document DE 103 24 646 A1 (corresponding to US 2004/0238019 A1). Spray nozzles are arranged in this cryostat, which are connected to a pump and through which cleaning or disinfection fluid is sprayed in program-controlled fashion in the interior of the cryostat.

With this type of automatic cleaning or disinfection as well, the temperature in the cryostat is elevated. As a result, the cryostat is unavailable for a long period for the processing of further specimens. A rapid cooling to working temperature and thus a rapid establishment of operational readiness can, if necessary, be compensated for by an elevated consumption of energy by the cooling device.

The document DE 103 52 575 A1 (corresponding to US 2005/0098563 A1) discloses a cryostat having an inner container, in which the inner container comprises a coating having soluble silver ions or the inner container is produced from a material that is doped with soluble silver ions. The antimicrobial action of the surface of the inner container depends on condensation of the inflowing ambient air. Here again, the best antimicrobial action is attained when the cryostat is defrosted and is no longer in a state ready for operation.

### SUMMARY OF THE INVENTION

It is therefore the object of the present invention to improve the cleaning and/or disinfection procedure inside the cryostat, and to ensure complete disinfection while operation proceeds.

This object is achieved, according to the present invention, by a cryostat wherein the disinfection device comprises a UV light source. Advantageous refinements of the invention are described herein.

The invention is characterized in that a UV light source is provided for disinfection. This light source can be switched on and off again at any desired point in time, in which context the cryostat need not be defrosted. The UV radiation guarantees complete disinfection of the cryostat with respect to fungi, yeasts, and bacteria, in a short period of time.

In a further embodiment of the invention, the UV light source is embodied as a UV-C light source, and is additionally arranged in the interior of the cryostat. The use of a UV-C light source results in a rapid (approx. 30 minutes) and reliable disinfection of cryostats. The light source is advantageously arranged or integrated in the interior of the cryostat. It has proven to be advantageous to arrange the UV-C light source directly above the cutting knife or specimen holder, since most contaminated material occurs there.

A particularly effective UV-C light source radiates light at a wavelength of lambda=254 nm. This radiation, which is close to X-rays, of course requires that various safety aspects be taken into account when operating the light source. The integration of the light source in the interior of the cryostat, and the arrangement of a safety switch in the closable cover of the cryostat, ensure that operation of the light source is possible only with the cover closed, and that the light source is immediately switched off by a control circuit upon opening of the cover.

In a refinement of the invention, the control circuit is equipped with an automatic time controller, so that automatic disinfection is performed at preselectable times. Because the cryostat does not need to be defrosted during the disinfection procedure, disinfection can be interrupted at any time and cutting of a frozen specimen can be performed.

In a refinement of the invention, the container is, or parts of the cryostat container are, equipped with a coating that contains titanium dioxide nanoparticles. A coating of this kind has the property of breaking down water into OH radicals, and at the same time forming hydrogen peroxide (H₂O₂) with atmospheric oxygen, by photocatalysis with the UV radiation. Both substances serve to disinfect the cryostat.

In a further embodiment of the invention, the coating additionally contains silver ion nanoparticles. The result of this is that silver ions (Ag⁺) are formed in interaction with water and contribute to disinfection of the cryostat.

### BRIEF DESCRIPTION OF THE DRAWINGS

The invention will be explained in more detail with reference to an exemplifying embodiment with the aid of the schematic drawings, in which:

FIG. 1 is a view of the cryostat; and

FIG. 2 is a view of the container in the cryostat.

### DETAILED DESCRIPTION OF THE INVENTION

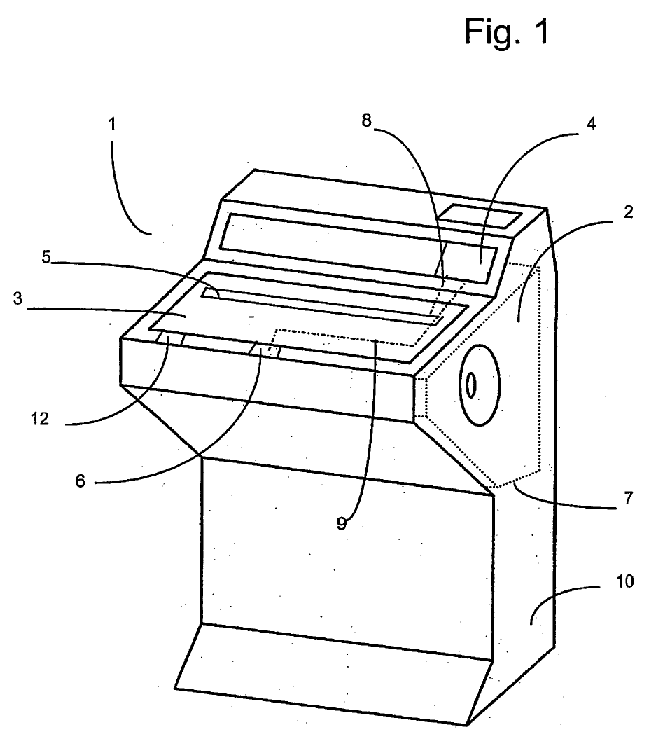

FIG. 1 is a view of cryostat 1 having a housing 10 and a container 2 arranged in housing 10. Container 2 serves to receive a microtome (not depicted) having a cutting knife. A UV-C light source 5 is fixedly arranged in the interior of the container and connected via an electrical line 8 to a control circuit 4. A cover 3 closing off container 2 is arranged on housing 10. Arranged between housing 10 and cover 3 is a safety switch 6 that is electrically connected via a line 9 to control circuit 4. Also associated with cover 3 is an electrically operating locking system 12.

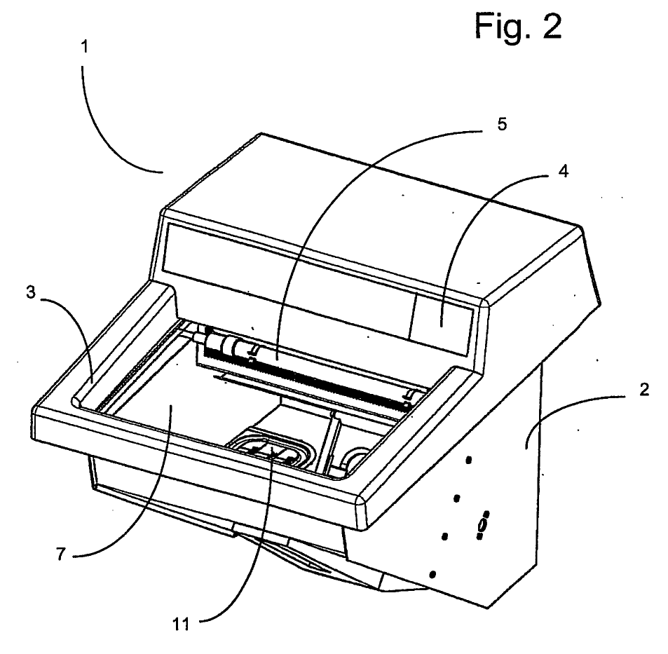

FIG. 2 shows container 2 having UV-C light source 5 arranged therein, and a cooling device 11. Container 2 comprises on its inner side a coating 7. Coating 7 contains titanium dioxide nanoparticles as well as, additionally, silver ion nanoparticles.

For disinfection of container 2, UV-C light source 5 is started manually or automatically, via a timer integrated into control circuit 4, for a preselected time span. It must be ensured in this context that cover 3 is closed. That state is ascertained via safety switch 6. A locking system 12 that electromechanically closes cover 3 is additionally provided in cover 3. Locking system 12 is electrically connected to control circuit 4.

As a result of safety switch 6 and locking system 12, operation of the UV-C light source is possible only with cover 3 closed.

After the preselected time span has elapsed, or by manual termination, UV-C light source 5 is switched off and locking system 12 is disengaged again.

### PARTS LIST

1 Cryostat
2 Container
3 Cover
4 Control circuit
5 UV-C light source
6 Safety switch
7 Coating
8 Electrical line 4-5
9 Electrical line 4-6
10 Housing
11 Cooling device
12 Locking system

What is claimed is:

1. A cryostat comprising:
a housing;
a container in the housing for receiving a microtome having a knife holder;
a cover operable to allow a user to gain access to an interior of the container and to close off the container;
a disinfection device associated with the container, the disinfection device including a UV light source; and
a control circuit connected to the disinfection device for activating and deactivating the disinfection device.

2. The cryostat according to claim 1, wherein the UV light source is a UV-C light source arranged in the interior of the container.

3. The cryostat according to claim 2, wherein the UV-C light source is located directly above a knife holder of a microtome when a microtome is received by the container.

4. The cryostat according to claim 2, wherein the wavelength radiated by the UV-C light source is 254 nanometers.

5. The cryostat according to claim 1I, further comprising a safety switch associated with the cover for ascertaining if the cover is opened or closed.

6. The cryostat according to claim 5, wherein the control circuit is electrically connected to the UV light source and to the safety switch.

7. The cryostat according to claim 6, wherein the control circuit has an integrated timer for automatically deactivating the UV light source after a preselected span of time.

8. The cryostat according to claim 6, wherein the UV light source is deactivated via the control circuit when the safety switch ascertains the cover is opened.

9. The cryostat according to claim 1, wherein at least a portion of the container comprises a coating having titanium dioxide nanoparticles.

10. The cryostat according to claim 9, wherein the coating (7) additionally has silver ion nanoparticles.

11. The cryostat according to claim 1, further comprises a locking apparatus arranged between the cover and the housing and electrically connected to the control circuit.

12. The cryostat according to claim 12, wherein the locking apparatus locks the cover in a closed state when the UV light source is activated.

13. A system comprising:
a housing;
a container in the housing;
a microtome received in the container, the microtome having a knife holder;
a cover operable to allow a user to gain access to an interior of the container and to close off the container;
a UV light source located in the container for disinfecting an interior of the container; and
a control circuit connected to the UV light source for activating and deactivating the disinfection device.

14. The system according to claim 13, wherein the UV light source is a UV-C light source.

15. The system according to claim 13, wherein the UV light source is located directly above the knife holder of the microtome.

16. The system according to claim 13, further comprising a safety switch associated with the cover for ascertaining if the cover is opened or closed.

17. The system according to claim 16, wherein the control circuit is electrically connected to the UV light source and to the safety switch, wherein the UV light source is deactivated by the control circuit when the safety switch ascertains that the cover is opened.

18. The system according to claim 13, wherein at least a portion of the container comprises a coating having titanium dioxide nanoparticles.

19. The system according to claim 13, further comprising a locking apparatus arranged between the cover and the housing and electrically connected to the control circuit, wherein the locking apparatus prevents the cover from being opened while the UV light source is activated.
